(19) United States
(12) Patent Application Publication
(10) Pub. No.: US 2004/0092929 A1
(43) Pub. Date: May 13, 2004

(54) Spinal plate with means to secure a graft

(76) Inventor: Michael R. Zindrick, Burr Ridge, IL (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/260,258

(22) Filed: Sep. 27, 2002

(51) Int. Cl. A61B 17/56

(52) U.S. Cl. 606/61

(57) ABSTRACT

Methods and apparatus are disclosed for securing grafts and fusing vertebrae within the spine following discectomy or vertebrectomy surgery. In one embodiment, a device for use after a discectomy comprises a plate having a curvature conforming to the natural anterior or lateral curvature of the cervical spine. The plate preferably has an inspection opening with a generally rectangular shape. The plate also has horizontally extending prongs disposed on opposite sides of the opening. A graft is inserted between the prongs. The upper end of the plate is secured to the vertebra above the removed disc and the lower end of the plate is secured to the vertebra below the removed disc. When the plate is secured to the spine, the prongs and graft extend into the space previously occupied by the removed disc. The graft is secured within the disc space by the prongs. The inspection opening in the implant facilitates visual observation by the implanting surgeon of the position of the graft relative to the adjacent vertebrae, and thus permits verification that the graft is secured in close physical contact with the vertebrae to ensure successful fusion. In another embodiment, a device for use after a vertebrectomy comprises a longer plate having a curvature conforming to the natural posterior curvature of the cervical spine with two inspection openings.

### BACKGROUND OF THE INVENTION

1. Field of the Invention

This invention relates generally to bone implants, and more particularly, to spinal implants for securing adjacent vertebrae to each other.

2. Description of the Related Art

Numerous factors, including trauma, tumors, infections and degenerative diseases can cause parts of the spinal column to develop fractures, outgrowths, or ridges that can restrict freedom of movement and cause extreme pain or even paralysis. Furthermore, intervertebral discs can become herniated or they can degenerate and shrink. When the discs herniate, they may pinch nerves or put pressure on the spinal cord. When discs degenerate and shrink, they cease to provide the proper spacing and necessary cushioning between adjacent vertebrae.

Those skilled in the art have developed a variety of approaches for treating these conditions, such as removing all or portions of intervertebral discs and vertebrae and, where appropriate, replacing the removed discs or vertebrae with a graft or implant. In some instances, the graft is taken from another site on the patient's body (autograft); in other instances, the graft may be obtained from a donor “bone bank” (allograft). Additionally, bone morphogenic protein impregnated substances and foam metal materials encouraging bony in-growth are sometimes used. Given the right conditions, each of the adjacent vertebrae in contact with the graft will fuse to the graft and become, in effect, one large unitary bone. The fused vertebrae will no longer move with respect to each other, thus limiting the person's flexibility. However, the fusion minimizes or eliminates the risk of further injury to the spinal column and spinal cord, and the pain caused by defects in the vertebrae or discs. To achieve a successful fusion, the spine must be stabilized so that the bones have time to fuse. The fusion process typically takes from about six weeks to about six months.

In order for a successful fusion to occur, the graft must be placed into physical contact with, and receive pressure from, the adjacent vertebrae. The graft and vertebrae must also be stabilized with respect to each other. Previous attempts to stabilize the vertebrae and grafts have had many deficiencies. For example, prior art implants have not adequately secured the graft between the vertebrae, have not properly provided for post-surgery shifting of the space between the vertebrae, and have not permitted the implanting surgeon to properly verify the extent of contact between the vertebrae and the graft.

### SUMMARY OF THE INVENTION

Methods and apparatus are provided for securing grafts and fusing vertebrae within the spine following discectomy or vertebrectomy surgery. The disclosed invention is particularly well suited for use on the cervical spine. In one embodiment, a device for use after a discectomy comprises a plate having a curvature conforming to the natural lordotic curvature of the cervical spine. The plate preferably has a large inspection opening with a generally rectangular shape. The plate also has horizontally extending prongs disposed on opposite sides of the opening. The upper and lower edges of each prong are preferably tapered such that the prongs have a trapezoidal shape when viewed from the side. The prongs may also be fenestrated to facilitate post-operative inspection of the space surrounded by the prongs.

A graft is inserted between the prongs (comprising, for example, a human bone fragment or an artificial bone substitute such as morphogenic protein impregnated substances or foam metal). The upper end of the plate is secured to the vertebra above the removed disc and the lower end of the plate is secured to the vertebra below the removed disc. When the plate is secured to the spine, the prongs and graft extend into the space previously occupied by the removed disc. The graft is laterally secured within the disc space by the prongs. The inspection opening and fenestrations in the prongs facilitate visual observation by the implanting surgeon of the position, orientation, and interface of the graft relative to the adjacent vertebrae, and thus permits verification that the graft is secured in close physical proximity (or in contact) with, and receives pressure from, the vertebrae to ensure successful fusion.

In another embodiment, a device for use after a vertebrectomy is similar to the discectomy device, but is longer and preferably comprises two inspection openings through which the surgeon verifies that the graft is secured in close physical proximity or contact with each of the adjacent vertebrae. In this embodiment, the horizontally extending prongs are also longer to help secure a larger graft between the space previously occupied by the removed vertebra.

### BRIEF DESCRIPTION OF THE DRAWINGS

FIG. 1 is a posterior perspective view of one embodiment of an implant for securing a graft within the spine.

FIG. 2A is an anterior plan view of the implant of FIG. 1.

FIG. 2B is a side elevational view of the implant of FIG. 1.

FIG. 2C is a top plan view of the plate of FIG. 1.

FIG. 3 is a side view of a normal cervical spine.

FIG. 4A is a side view of the cervical spine of FIG. 3 after an intervertebral disc has been removed and the implant of FIG. 1 has been inserted to secure a graft between two adjacent vertebrae.

FIG. 4B is an anterior view of the cervical spine and the implant as shown in FIG. 4A.

FIG. 5A is a posterior view of an alternative embodiment of an implant for securing a graft within a cervical spine.

FIG. 5B is a side elevational view of the implant of FIG. 5A.

FIG. 5C is a top plan view of the implant of FIG. 5A.

FIG. 6 is a top plan view of another alternative embodiment of an implant for securing a graft within the spine.

FIG. 7A is an anterior view of an alternative embodiment of an implant for securing a graft within the spine following removal one or more vertebrae.

FIG. 7B is a side elevational view of the plate of FIG. 7A.

FIG. 7C is a top plan view of the implant of FIG. 7A.

FIG. 8A is a lateral view of the cervical spine of FIG. 3 with the implant of FIG. 7A securing a graft between two vertebrae after an adjoining vertebra has been removed.

FIG. 8B is an anterior view of the cervical spine and the implant of FIG. 8A

### DETAILED DESCRIPTION OF THE PREFERRED EMBODIMENTS

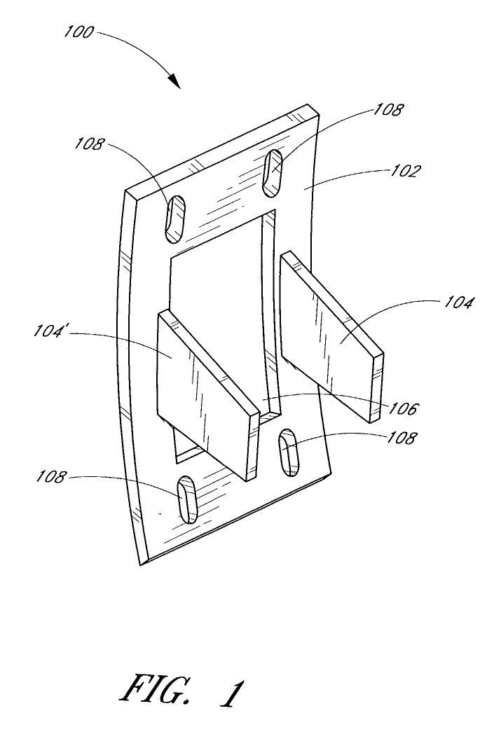

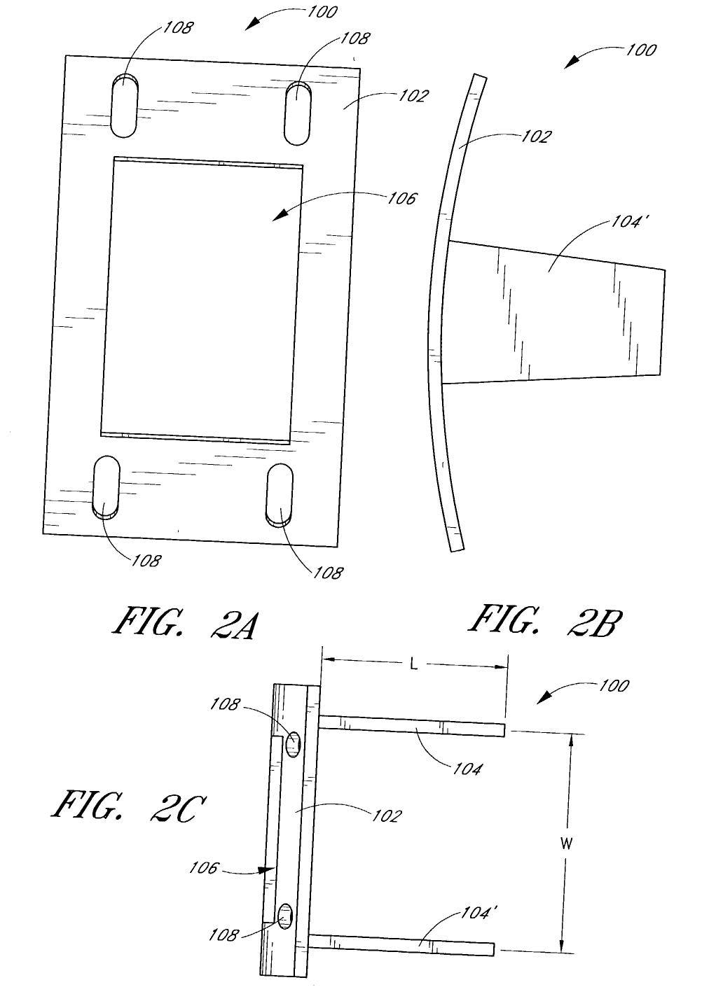

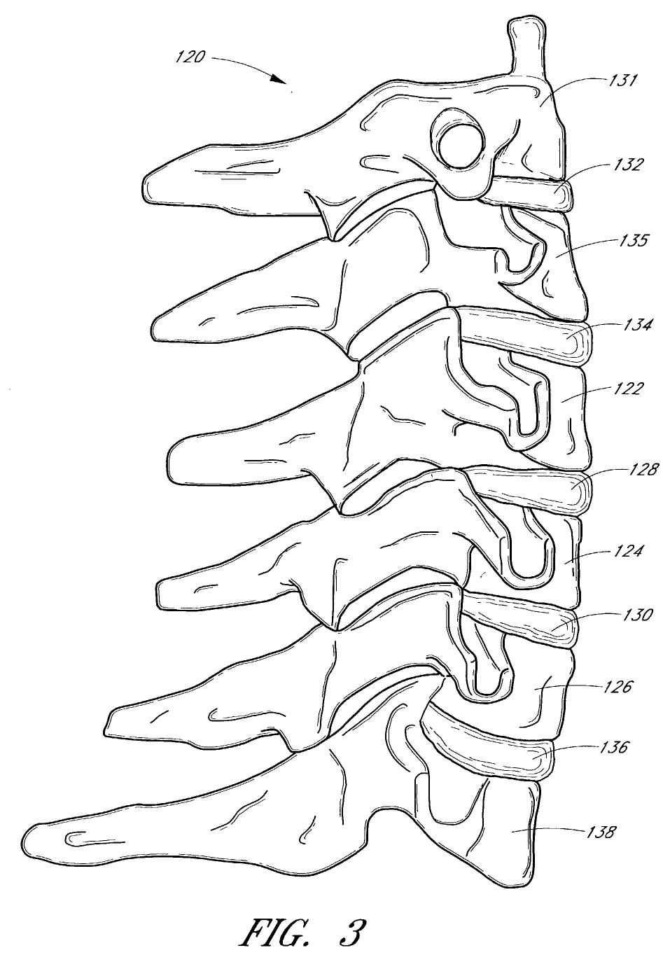

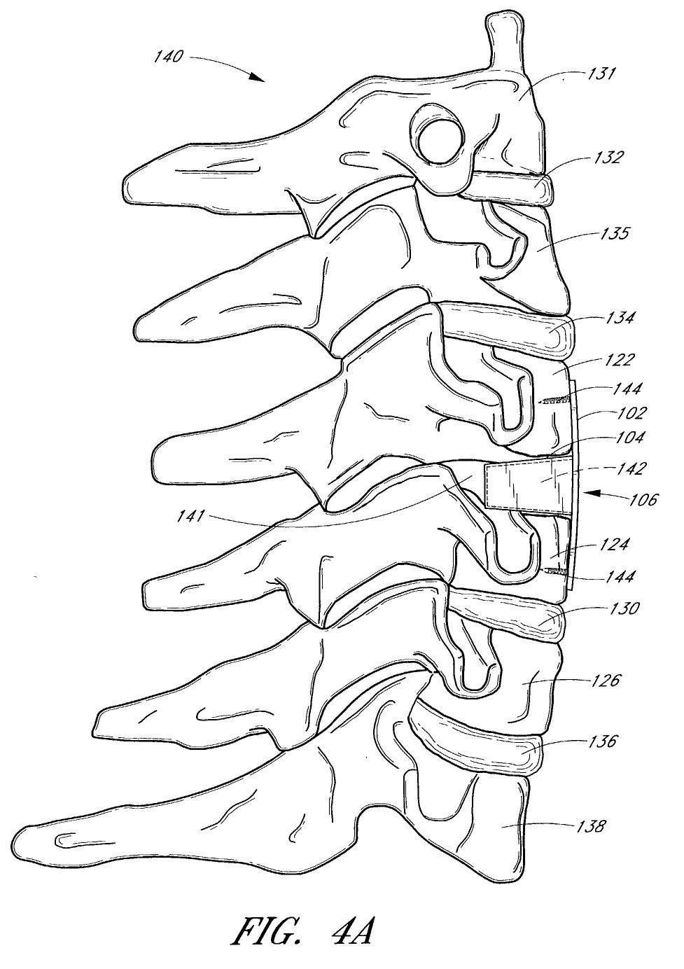

FIGS. 1 through 2C illustrate a preferred embodiment of an implant 100 adapted for securing a graft within the spine 120 (see FIG. 3). The implant 100 is preferably made of a biocompatible metal such as titanium or a composite material such as resin-impregnated carbon fiber, but could alternatively be made of any other biocompatible or resorbable material capable of withstanding the forces that act upon it after implantation within the spine. The implant 100 is especially well suited for use on the anterior of the cervical spine, but it may also be adapted in accordance with the principles of the present invention for use anywhere along the length of the spinal column, including the anterior of the cervical spine, or the anterior or lateral positions along the thoracic or lumbar spine.

As shown in FIGS. 1 and 2A, the cervical implant 100 comprises a base 102 with a vertical curvature such that the base 102 substantially conforms to the posterior curvature of the spine 120. The base 102 preferably has a large, generally rectangular inspection opening 106. The base 102 further comprises fastener holes 108. In a preferred embodiment, each of the holes 108 has a lengthened vertical dimension. A pair of prongs 104, 104′ are disposed on the anterior side of the base 102 near opposite edges of the opening 106, and may be fenestrated to facilitate inspection or assessment of the implanted graft after insertion.

As shown in FIG. 2B, the upper and lower edges of the prongs 104, 104′ are preferably tapered inwardly such that each prong has a trapezoidal shape when viewed from the side. As illustrated in FIG. 2C, the prongs 104, 104′ each have a length dimension L and are separated by a width dimension W. As explained in more detail below, the length and width dimensions L and W, are chosen so as to provide an optimally sized contact area between the vertebrae and a graft, based on the position within the spine 120 wherein the cervical implant 100 is intended to be used. As shown, the prongs 104, 104′ are preferably, though not necessarily, made of a solid piece of material rather than, for example, a mesh or perimeter structure, to provide sufficient rigidity and structural integrity to withstand the vertical and lateral forces that act upon them.

The dimensions of the implant will vary depending upon, for example, the area of the spine being treated, the size of the person being operated upon, and the composition of the implant. After considering each of these factors, and performing measurements on the patient, the surgeon preferably has a wide range of implant sizes from which to select for a given patient. By way of example, a representative size for the base 102 is about 26 mm by 20 mm. On the same implant 100, a representative size for the inspection opening is about 18 mm by 12 mm, a representative length (L) for the prongs 104, 104′ is about 12 mm, and a representative width (W) between prongs 104, 104′ is about 15-20 mm. Moreover, the radius of curvature of the base 102 will vary depending on where in the spine the implant 100 is used. For example, the implant location may affect whether the base 102 is concave or convex in the posterior direction when viewed in side elevation. Many other dimensions are contemplated and encompassed by the present invention. For example, implants of the present invention may have varying dimensions from those set forth above to accommodate different sizes and levels of vertebrae.

FIG. 3 is a lateral view illustrating a normal cervical spine 120. As is well known in the art, the cervical portion of the spine comprises seven vertebral bodies separated by intervertebral discs. FIG. 3 illustrates the lower six vertebrae of the cervical spine 120. As shown, the cervical spine 120 comprises vertebral bodies 131, 135, 122, 124, 126, 138 and adjoining intervertebral discs 132, 134, 128, 130, 136. The vertebral body 131 represents the second cervical vertebra (C2), the vertebral body 135 represents the third cervical vertebra (C3), and the vertebral body 122 represents the fourth cervical vertebra (C4). Similarly, the vertebral body 124 corresponds to the fifth cervical vertebra (C5), the vertebral body 126 corresponds to the sixth cervical vertebra (C6), and the vertebral body 138 corresponds to the seventh, and final, cervical vertebra (C7). The intervertebral disc 132 is disposed between the vertebral bodies 131, 135, the disc 134 is disposed between the vertebral bodies 135, 122 and the disc 128 is disposed between the vertebral bodies 122, 124. Similarly, the disc 130 is disposed between the vertebral bodies 124, 126, and the disc 136 is disposed between the vertebral bodies 126, 138.

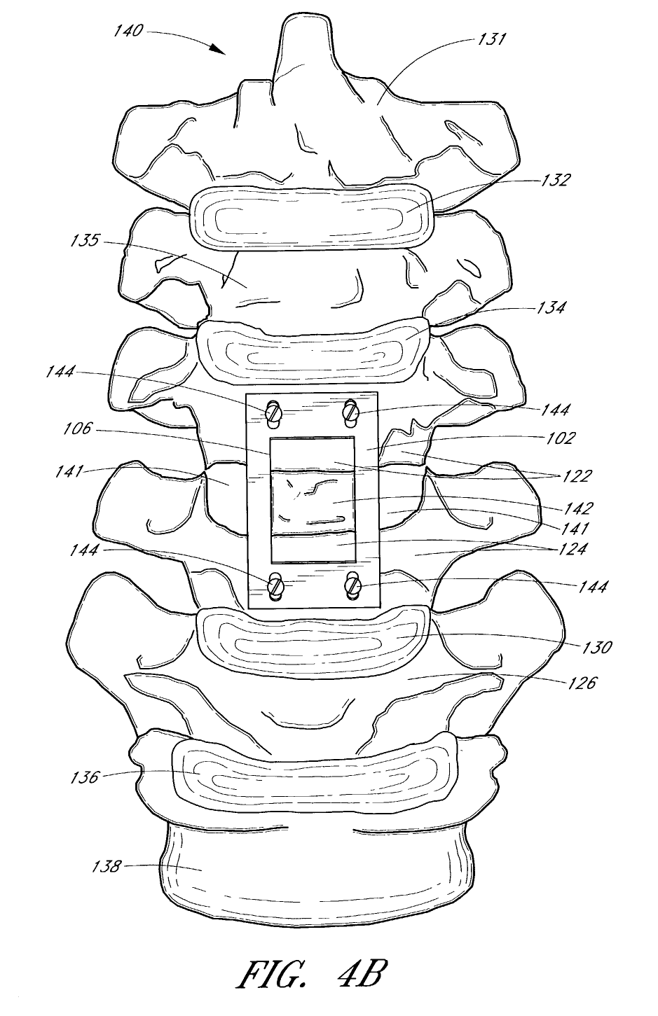

FIGS. 4A and 4B illustrate an exemplary use of the implant 100 following a discectomy performed in the cervical spine 140. FIG. 4A provides a lateral view of the treated cervical spine 140, whereas FIG. 4B provides an anterior view of the treated cervical spine 140. Before inserting the implant 100, a surgeon gains access to the cervical spine and removes the damaged or diseased disc. Once the disc has been removed, there is a void between the adjacent vertebrae. The void is filled and the vertebrae are stabilized by securing the implant 100 and a graft 142 between the adjacent vertebrae.

A suitably sized graft 142 is obtained and positioned between the prongs 104, 104′ of the implant 100 (see FIGS. 1 and 2C). The graft 142 is preferably comprised of a human bone fragment or an artificial bone substitute such as morphogenic protein impregnated substances or foam metal. Those of skill in the art will recognize after reading this disclosure that other types of grafts are contemplated and encompassed by this invention. The graft must be appropriately sized. If the graft is too small, it will not properly interface with the adjacent vertebrae, and the likelihood of a successful fusion is greatly diminished. An unduly small graft may also migrate from the desired location to a different location where it would be less effective, or could even cause serious injury or inflammation. On the other hand, the graft must be large enough to fit snugly between the adjacent vertebrae, but not so large as to exert a substantial force against the vertebrae such that the natural location or orientation of the vertebrae would be disrupted.

The size and distance between the prongs 104, 104′ are chosen so that a graft 142 of appropriate size can be inserted between and be firmly held by the prongs 104, 104′ on the base 102. In this way, the graft 142 is not likely to slide out from between the prongs 104, 104′ during the insertion step, or to migrate from the desired location during the surgery or during the fusion process over the weeks that follow. The prongs 104, 104′ thus facilitate the insertion and fixation of the graft and enhance the spinal stabilizing characteristic of the implant 100. To achieve an optimal fit between the graft 142 and the prongs 104, 104′ of the implant 100, the physician may scrape away or otherwise remove additional material from the graft 142 before inserting it between the vertebrae 122, 124.

The implant 100 and graft 142 is then inserted into the space 141 between the adjacent vertebrae 122, 124. The tapering of the prongs 104, 104′ facilitates insertion of the prongs 104, 104′ into the space between the vertebrae 122, 124. Because the width of the anterior edges of the prongs 104, 104′ is narrower than the width of their posterior edges, the prongs 104, 104′ can be inserted into smaller disc spaces 141. In fact, as the implant 100 is pushed into the disc space, the wider posterior edges of the prongs 104, 104′ progress further into the disc space and may even, if necessary, spread the adjacent vertebrae further apart (depending upon the selected size of the implant 100).

Moreover, even if it is not necessary to spread the vertebrae further apart, the natural spacing between the vertebrae is generally somewhat wider near the posterior side and somewhat narrower near the anterior side, and hence the tapering permits the prongs 104, 104′ to be as wide as possible to provide contact and support across the surfaces of the upper and lower vertebrae 122, 124 without exerting undue force against the vertebrae 122, 124. In this way, the tapered prongs 104, 104′ serve to achieve the proper lordosis, or orientation, of the vertebrae 122, 124 with respect to each other, preserving the natural and desirable curvature of the spine.

The inspection opening 106 on the implant 100 facilitates visual observation of the position of the graft 142 relative to the vertebrae 122, 124 between which the graft 142 has been inserted. Fenestrations in the prongs 104, 104′ would also facilitate visual observation of the position of the graft 142 relative to the vertebrae 122, 124. A newly secured graft 124 must be placed under pressure and in close physical proximity or contact with the adjacent vertebrae 122, 124 in order for successful bone fusion to occur. To ensure that close physical proximity or contact is established between the graft 142 and the vertebral bodies 122, 124, the surgeon looks through the opening 106 in the base 102 (see FIG. 4B) to observe the lines of contact between the graft 142 and the vertebral bodies 122, 124. Moreover, the visual inspection may reveal that the graft 142 is either too large or too small and the surgeon can then withdraw the implant 100 and use a different size of graft.

The inspection opening 106 is preferably generally rectangular in shape. A rectangular opening will usually provide the largest amount of viewing area along each of the upper and lower lines of interface between the graft 142 and the adjacent vertebrae 122, 124. A wide viewing area is desirable because it allows the surgeon to inspect nearly the full length of the interface to verify that the graft 142 and the vertebrae 122, 124 are in close proximity or contact. In addition, the surgeon's visual inspection of the interface between the graft 142 and the vertebrae 122, 124 will also influence the surgeon's determination of where to secure the upper and lower edges of the implant 100 to the posterior sides of the respective vertebrae 122, 124 (as more fully explained below). Other opening shapes may also function well, such as squares, ovals, circles, or any other opening that provides adequate viewing area along the interface between the graft 142 and vertebrae 122, 124.

The inspection opening 106 and prong fenestrations also provide benefits during radiographic viewing procedures (such as X-ray fluoroscopes or films). As previously explained, one preferred material for the implant 100 is a biocompatible metal material. Metals often produce bright spots on X-rays and thus obscure the portions of the body behind the metal. The inspection opening 106 and prong fenestrations advantageously allow the surgeon to view the graft 142 and the affected portion of the vertebrae 122, 124 without obstruction by the implant during the implantation procedure and during the recovery period.

As shown in FIG. 4B, after the implant 100 and graft 142 have been inserted between the adjacent vertebrae 122, 124, and the surgeon has verified proper interface between the graft 142 and the vertebrae 122, 124, the surgeon then passes fasteners 144 through the holes 108 on the upper and lower sides of the implant 100 and into the posterior sides of the respective vertebrae 122, 124.

In a preferred embodiment, as shown, there are four holes 108. Two of the holes 108 are positioned along the upper edge of the implant 100 and two of the holes 108 are positioned along the lower edge of the implant 100. Preferably, the upper holes are each positioned the same distance from the top edge of the implant 100 and the lower holes are each positioned the same distance from the bottom edge of the implant 100. In addition, each of the upper and lower holes are preferably located the same distance away from the vertical centerline of the implant 100. By positioning the holes in a symmetrical configuration as shown and described, each fastener 144 positioned in a hole 108 bears approximately the same amount of load after the implant 100 is inserted and secured to the vertebrae 122, 124. Any number of other hole configurations may also provide adequate load-bearing capability. In addition, the screws may include locking mechanisms to prevent them from reverse rotating and possibly withdrawing from the surrounding bone and the implant 100.

The fasteners 144 are preferably appropriately sized screws. The surgeon must carefully select the points of attachment of the base 102 on the vertebrae 122, 124 to ensure that the vertebrae 122, 124 exert sufficient pressure on the graft 142 to promote bone growth and fusion, and to properly orient the vertebrae 122, 124 with respect to each other.

The holes 108 are preferably elongated to provide some degree of freedom of movement in the event that the spine 120 expands or lengthens slightly during the fusion and healing process. The fasteners 144 are inserted with sufficient tightness that the implant 100 cannot freely slide up and down, but will, under a substantial force, move a small distance. In this way, the implant 100 will accommodate the small amount of natural shifting that sometimes occurs during the fusion process between the graft 142 and the adjacent vertebrae 122, 124. The elongated holes 108 decrease the likelihood that this shifting between the vertebrae 122, 124 and graft 142 will cause the fasteners 144 to dislodge from the vertebrae or that the plate 102 will bend or twist.

After the surgeon inserts the prongs 104, 104′ between the vertebrae 122, 124 and secures the plate 102 to the vertebrae 122, 124, the surgeon closes and dresses the incision. The vertebral bodies 122, 124 will then, in time, grow into and integrate with the implant 100 and graft 142, forming, in effect, one large unitary bone. The fusion of the vertebral bodies 122, 124 supports the spine 140 and prevents the collapse of the intervertebral space 141, which would otherwise occur in absence of the removed intervertebral disc 128. The fusion also prevents motion of one or more of the vertebrae that are causing pain, and resolves spinal instabilities arising from tumors, infections, trauma, or disease.

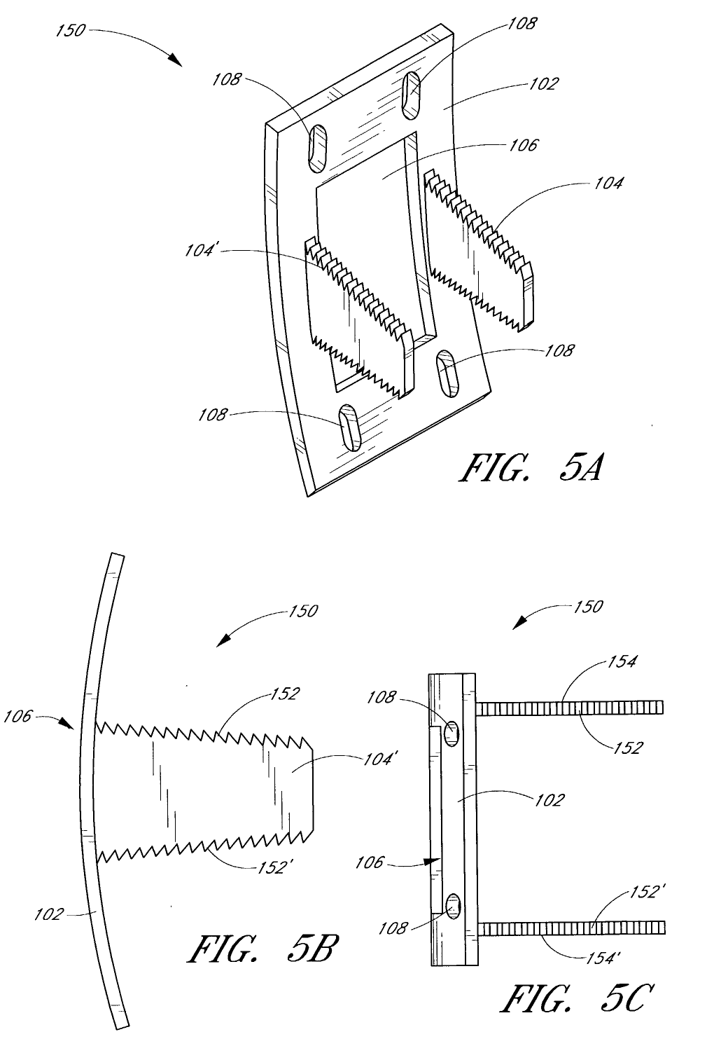

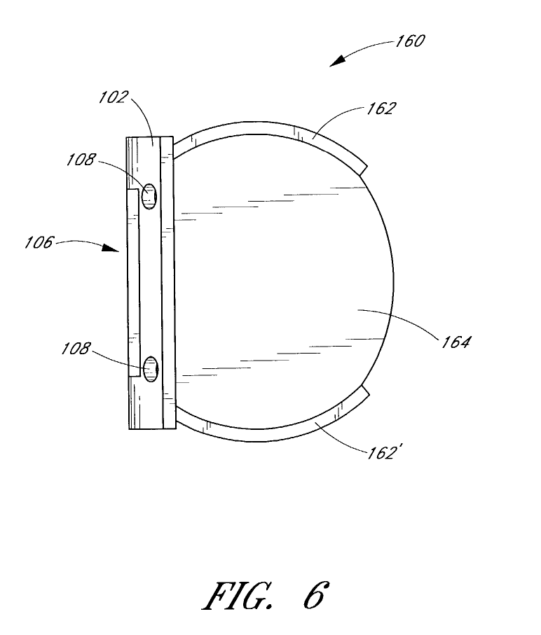

FIGS. 5A through 5C illustrate an alternative embodiment of an implant plate 150 for securing a graft 142 within the spine 140. The implant 150 is similar to the implant 100 of FIGS. 1 through 2C, except that the prongs 154, 154′ of implant 150 each have roughened upper and lower edges. In the example shown in the figures, the prongs 154, 154′ have sawtooth edges 152, 152′. The individual teeth of the sawtooth edges preferably lean toward the posterior side of the implant 150 as shown. When the prongs 154, 154′ are inserted between the two vertebral bodies, the sawtooth edges 152, 152′ provide increased surface contact with the vertebral bone and the pointed tips of each individual tooth penetrates a very small distance into the vertebral bone. In this way, the prongs 154, 154′ provide a more secure grip on the respective vertebral surfaces. The anterior leaning of the individual teeth makes it relatively easy to slide the implant 150 into place by pushing it in the posterior direction, but provides substantial resistance against unintended migration of the implant in the posterior direction after insertion. Roughened surfaces may also be provided on other surfaces of the implant 150, such as the lateral sides of the prongs 154, 154′ and/or the posterior side of the base 102 to help secure the implant 150 to the vertebrae and to promote bone fusion.

The sawtooth edges 152, 152′ also serve to help hold the implant 150 in place while the surgeon passes the fasteners 144 through the implant 150 and into the anterior sides of the respective vertebrae 122, 124. Any number of other roughened surfaces also could be used to provide increased surface area contact and to diminish the likelihood of unintended migration of the implant 150. For example, the edges of the prongs 154, 154′ could be formed with bumps, knurling, cross-hatching, vertical projections, or similar roughened surfaces. After insertion of the implant, as the graft 142, vertebrae, and implant 100 fuse together, the roughened surfaces on the edges of the prongs 154, 154′ also provide more surface area on which new bone cells can gather and grow. This expedites the fusion process and reduces the chance that the implant 150 may loosen, dislodge, or migrate from its intended location.

FIG. 6 illustrates another embodiment of an implant 160 for stabilizing and fusing adjacent vertebrae. The implant 160 of FIG. 6 is similar to the implant 100 of FIGS. 1 through 2C, except that the prongs 162, 162′ of the implant 160 are horizontally curved to more securely hold a rounded graft 164. Because the curved prongs 162, 162′ bow out laterally beyond where the generally straight prongs 104, 104′ of FIG. 1, the curved prongs 162, 162′ is able to hold a larger graft 164 that provides increased surface area contact between the graft 164 and the vertebral bodies 122, 124, thereby yielding greater spinal stability as well as enhancing the likelihood of a successful fusion. In addition, because the space between the curved prongs 162, 162′ on the anterior side is much smaller than the thickest portion of the graft 164, the graft 164 is less likely to migrate in an anterior direction away from the implant 160. Moreover, after completion of the fusion process, the implant 160 with curved prongs 162, 162′ is more securely bound to the graft 164 and adjacent vertebrae 122, 124.

As with the graft 142, the graft 164 may be either an autograft or an allograft. When the original or “harvested” shape of the graft 164 is not round, the graft 164 may be shaved or otherwise manipulated so that the graft 164 fits between the rounded prongs 162, 162′. Moreover, roughened surfaces, such as sawtooth edges 152, 152′ of the implant 150, may be incorporated into the rounded prongs 162, 162′ of the implant 160 to further enhance the bone-gripping feature of the implant 160.

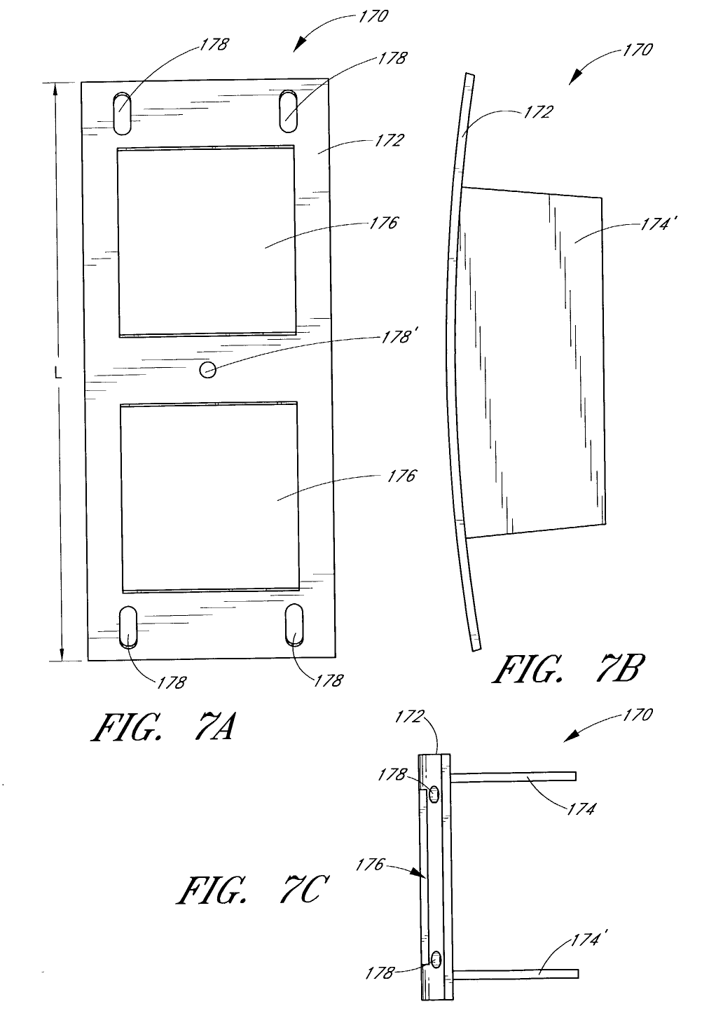

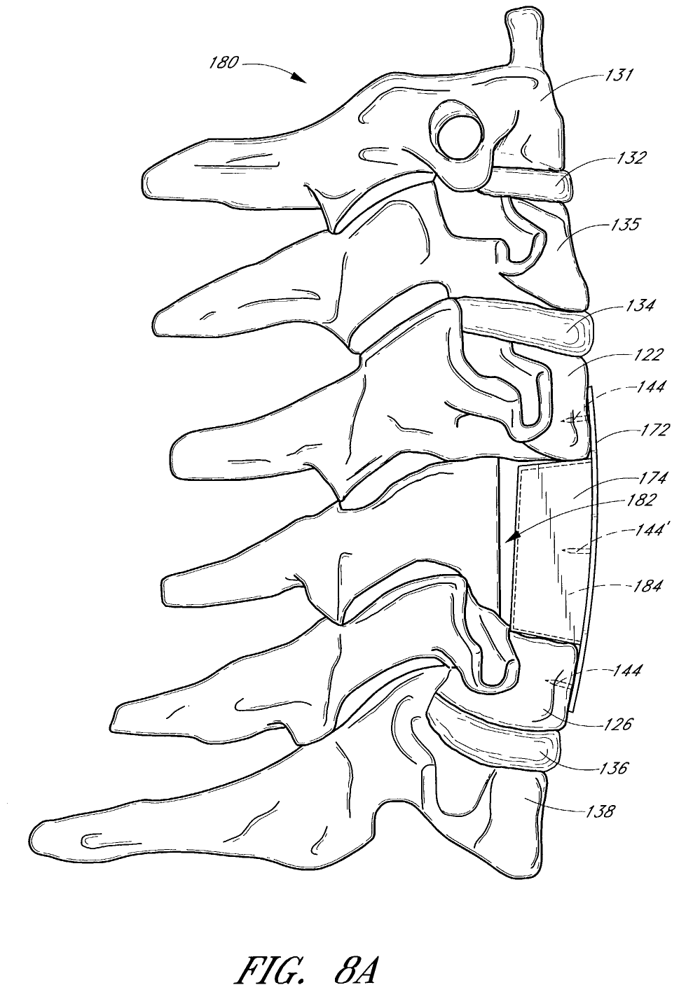

FIGS. 7A through 7C illustrate another alternative embodiment of an implant 170 which can be used for stabilizing and promoting fusion in the spine following removal one or more vertebrae (vertebrectomy). The concepts and advantages previously described in connection with the foregoing embodiments apply equally to implant 170. Although implant 170 is described herein with specific reference to the cervical portion of the spine, the implant 170 may be adapted for use anywhere along the spine, including anterior or lateral positions along the thoracic spine and the lumbar spine.

In the illustrated embodiment, the implant 170 comprises a base 172 which preferably has a vertical curvature such that the base 172 conforms to the anterior or lateral curvature of the spine. The implant 170 has a length dimension L which corresponds to the number of vertebrae to be removed. For example, in the case where only one vertebral body is to be removed, the length dimension L may be about 45 mm. In an alternative embodiment intended for use when multiple vertebrae have been removed, the length dimension L would be increased accordingly.

A pair of prongs 174, 174′, are disposed on the anterior side of base 172 on opposite sides of a pair of openings 176, 176′. As shown in FIG. 7B, the prongs 174, 174′ are tapered (as with the prongs 104, 104′ of implants 100) to facilitate insertion between the remaining upper and lower vertebrae 122, 126. The prongs 174, 174′ are preferably, though not necessarily, made of a solid piece of material rather than, for example, a mesh or perimeter structure, to provide sufficient rigidity and structural integrity to withstand the vertical and lateral forces that act upon them. Alternatively, the prongs 174, 174′ may be fenestrated to facilitate inspection of the interior space of the implant after insertion.

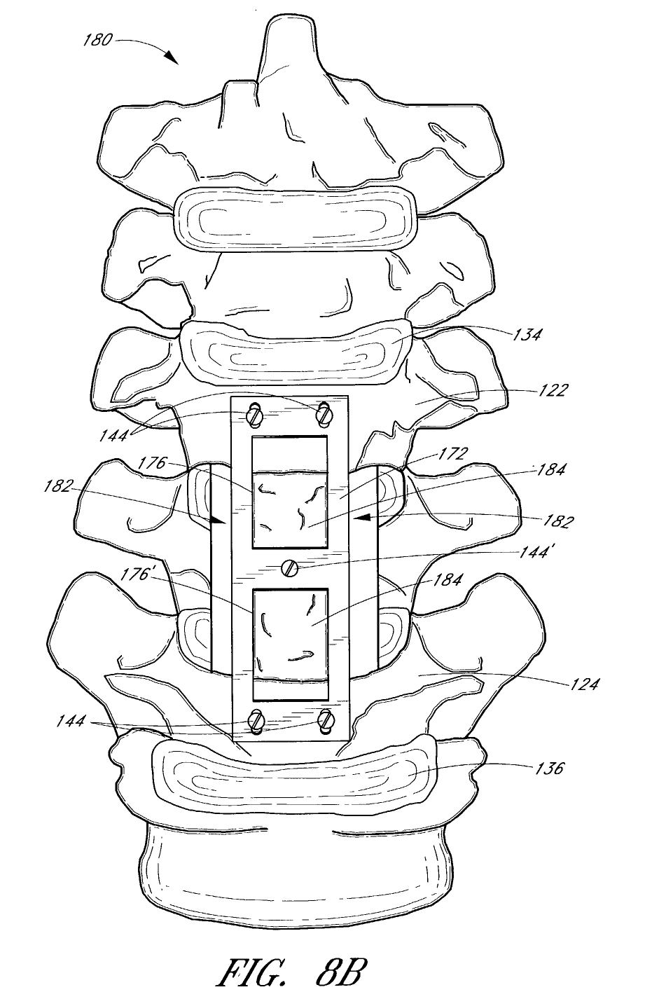

The prongs 174, 174′ serve to grasp a bone strut graft 184 (see FIG. 8B). A strut graft 184 is similar to the graft 142, except that the strut graft 184 is longer (for example, about 35 mm), so as to support the remainder of the spine after the removal of one or more vertebrae. In another alternative embodiment, the prongs 174, 174′ may be horizontally curved in a manner similar to the curved prongs 162, 162′ illustrated in FIG. 6. The prongs 174, 174′ may also comprise edges with roughened surfaces, such as the sawtooth edges 152, 152′ described in connection with implant 150 and illustrated in FIGS. 5A through 5C. Roughened surfaces may also be provided on other surfaces of the implant 170, such as the lateral sides of the prongs 174, 174′ and/or the anterior side of the base 172 to help secure the implant 170 to the vertebrae and to promote bone fusion.

As shown in FIG. 7A, the openings 176, 176′ facilitate visual observation of the position of the strut graft relative to the vertebrae between which the strut graft is inserted. As previously discussed, a newly obtained strut graft must be placed into physical contact with and receive compression forces from adjacent vertebrae in order for a successful fusion to occur. The openings 176, 176′ enable the surgeon to visually verify that a newly secured strut graft is in close physical proximity or contact with adjacent vertebrae.

The plate 172 further comprises holes 178, which are preferably elongated, and a hole 178′, which is preferably circular. The circular hole 178′ facilitates using a fastener 144′ (see FIG. 8B) to secure the long strut graft to the plate 172, while the elongate holes 178 facilitate using fasteners 144 to secure the plate 172 and the strut graft to the appropriate location on the respective upper and lower remaining vertebrae 122, 126 of the spine. The fasteners may be screws or other similar devices known to those of skill in the art.

Each of the elongate holes 178 is preferably oriented lengthwise parallel with the length dimension L of the plate 172. As with the holes 108 of implant 100, the holes 178 of implant 170 provide some degree of sliding movement under a substantial force between the fasteners and the plate 172 in the event that the spine expands or lengthens slightly during the fusion and healing process. Moreover, as with the holes 108 of the implant 100, the holes 178 are preferably arranged in a symmetrical configuration to properly spread the bearing load across the fasteners 144.

FIGS. 8A and 8B illustrate the implant 170 after being inserted with a strut graft 184 into the spine 180 following a vertebrectomy. In the illustrated example, a posterior cervical vertebrectomy has been performed to surgically remove one or more vertebrae that are pinching nearby nerves and causing pain (i.e., cervical stenosis). Once the posterior cervical spine 180 is accessed, a discectomy is performed on the intervertebral discs directly above and below the vertebral body to be removed. The target vertebral body is then removed from the spine 180, thus creating a space 182 between the vertebral bodies 122, 126.

With the vertebral body 124 removed from the spine 180, the vertebral bodies 122, 126 must remain in their current locations and be fused to support the spine 180 and prevent collapse of the resulting void 182. This is accomplished by securing a suitably sized strut graft 184 between the vertebral bodies 122, 126. The strut graft 184 is positioned between the prongs 174, 174′ of the cervical plate 170 (see FIGS. 7A and 7C). The strut graft 184 is sized such that the prongs 174, 174′ securely grip the strut graft 184. The strut graft 184 and the prongs 174, 174′ are then inserted into the space 182. The plate 172 is secured to the vertebral bodies 122, 126 by passing fasteners, such as appropriately sized screws 144, through the elongate holes 178 into the vertebral bodies 122, 126.

The strut graft 184 is preferably in close physical proximity or contact with, and under pressure from, both the vertebral bodies 122, 126 in order for successful fusion to occur. As mentioned above, the openings 176, 176′ in the plate 172 enable the physician to visually observe the interfaces between the strut graft 184 and the vertebral bodies 122, 126, as illustrated in FIG. 8B. This enables the physician to visually verify, during the cervical fusion surgery, and in radiographic procedures (such as X-rays) during and after the surgery, that the strut graft 184 is optimally positioned proximal to or in contact with the vertebral bodies 122, 126, thereby substantially increasing the likelihood that a successful fusion will occur. After insertion of the implant 170 and strut graft 184, the surgeon closes and dresses the incision. The surrounding vertebrae ultimately fuse with the strut graft 182 and the implant 170, which effectively results in a long unitary bone.

While the foregoing description sets forth various embodiments and details relating to preferred embodiments, it should be appreciated that the description is illustrative only and should not to be construed as limiting the invention. Of course, the specified dimensions may vary considerably depending upon the location or space between the adjacent vertebrae and individual patient variations. Thus, the scope of this disclosure is not to be limited by the illustrations or the foregoing descriptions thereof, but rather solely by the appended claims.

What is claimed is:

1. A spinal implant, comprising:
a curved plate having a posterior side and an anterior side, the plate having at least two elongated upper fastening holes and at least two elongated lower fastening holes, and a generally rectangular inspection opening; and
at least two tapered prongs on the plate which extend in a posterior direction away from the plate.

2. The spinal implant of claim 1, wherein the prongs include surface roughenings on at least one edge.

3. The spinal implant of claim 2, wherein the surface roughenings comprise a sawtooth pattern.

4. The spinal implant of claim 3, wherein the sawtooth pattern comprises a plurality of teeth which lean in the anterior direction.

5. The spinal implant of claim 1, wherein the prongs are curved.

6. The spinal implant of claim 1, wherein the upper fastening holes are each approximately the same distance from the upper edge of the plate, and the lower fastening holes are each approximately the same distance from the lower edge of the plate

7. The spinal implant of claim 6, wherein the fastening holes are each approximately the same distance from a vertical centerline of the plate.

8. A spinal implant, comprising:
a plate having a posterior side and an anterior side, the plate having a plurality of fastening holes and an inspection opening; and
at least two prongs on the posterior side of the plate which extend in a posterior direction away from the plate, each prong being formed of a solid piece of material.

9. The implant of claim 8, wherein the prongs have tapered upper and lower edges.

10. The implant of claim 9, wherein the fastening holes are elongated.

11. The implant of claim 10, wherein the inspection opening is generally rectangular.

12. The implant of claim 11, wherein the prongs are curved and include surface roughenings on at least one edge.

13. The implant of claim 8, wherein the plate is curved.

14. The spinal implant of claim 8, wherein the fastening holes comprise upper fastening holes and lower fastening holes, the upper fastening holes each being approximately the same distance from the upper edge of the plate, and the lower fastening holes each being approximately the same distance from the lower edge of the plate.

15. The spinal implant of claim 14, wherein the fastening holes are each approximately the same distance from a vertical centerline of the plate.

16. A method of stabilizing the spinal column, comprising:
obtaining a graft;
obtaining an implant plate with a posterior side and an anterior side, the plate having a plurality of fastening holes, at least one inspection opening, and at least two prongs which extend in the posterior direction away from the plate;
inserting the graft between the prongs;
inserting the implant plate and the graft into the space between two adjacent vertebrae; and
passing fasteners through the fastening holes and into the vertebrae to secure the implant to the vertebrae.

17. The method of claim 16 further comprising the step of passing fasteners through the implant plate and into the graft to secure the graft to the plate.
